(19) United States
(12) Patent Application Publication
(10) Pub. No.: US 2023/0026214 A1
(43) Pub. Date: Jan. 26, 2023

(54) DEVICE FOR MONITORING AN OXIDATIVE STRESS AND METHODS THEREOF

(71) Applicant: THE UNIVERSITY OF ADELAIDE, Adelaide, South Australia (AU)

(72) Inventor: Roman KOSTECKI, Adelaide, South Australia (AU)

(21) Appl. No.: 17/783,925

(22) PCT Filed: Dec. 9, 2020
(86) PCT No.: PCT/AU2020/051340

§ 371 Date: Jun. 9, 2022

(30) Foreign Application Priority Data
Dec. 9, 2019 (AU) 2019904655

(51) Int. Cl. G01N 21/64; G01N 33/52; G01J 3/44; C08L 37/00

(52) U.S. Cl. CPC G01N 21/64; G01N 33/52; G01J 3/4406; C08L 37/00

(57) ABSTRACT

The present relates, in general terms, to a device for monitoring oxidative stress in a sample, a method of making the device and a method of monitoring oxidative stress in a sample thereof.

### TECHNICAL FIELD

The present relates, in general terms, to a device for monitoring oxidative stress in a sample, a method of making the device and a method of monitoring oxidative stress in a sample thereof.

### BACKGROUND

Oxidative stress occurs when there is an imbalance between formation and regulation of reactive oxygen species (ROS) in cells and tissues, causing ROS levels to build-up and increase dramatically. This imbalance leads to damage of important biomolecules and cells, with potential impact on the whole organism. Oxidative stress influences many physiological processes including the immune system and cellular communication, and has been linked to intense exercise, inadequate diet, ageing and several age-related disorders, as well as many chronic diseases. Some of the chronic diseases shown to be associated with increased levels of oxidative stress include; cardiovascular diseases, including vascular diseases, high cholesterol, stroke, heart failure, and hypertension; cancer; Parkinson's disease; Alzheimer's disease; diabetes; kidney disease; rheumatoid arthritis; sepsis; and respiratory distress syndrome.

Protein carbonyls (aldehydes and ketones) are an example of a marker of global oxygen metabolism and oxidative stress, as they are generated by multiple different reactive oxygen species in blood, tissues, and cells. Protein carbonyls are abundant in plasma, chemically stable, and may be detected using a wide variety of laboratory-based analytical techniques. These methods for measurement of carbonyl content in biological samples were developed in the early 1970s and are still applied today. The main methods range from simple spectrophotometric analysis to liquid chromatography and mass spectrometry. However, all the existing methods are in-vitro and require careful sample handling and preparation and reported protein concentrations differ considerably depending on the applied protocols. For example, ELISA is considered the best available method to quantify protein carbonyl concentrations, whereas immunoblotting allows comparable detection of the molecular weight of oxidized proteins. These methods are widely used for the interpretation of changes in redox balance due to exercise, diabetes, cellular damage, aging, and age-related disorders. While these methods for measuring protein carbonylation have been implemented in different laboratories around the world, to date no methods prevail as the most accurate, reliable, and robust.

Further, all the existing methods for measuring protein carbonylation require some form of sample preparation. These methods yield information that is static in nature and therefore unable to measure dynamic changes in protein carbonylation.

It would be desirable to overcome or ameliorate at least one of the above-described problems, or at least to provide a useful alternative. In particular it would be useful to have a device capable of repeatedly measuring the oxidative stress state of a biological system and in particular improve upon prior devices for oxidative stress detection, which requires sample preparation in all cases and complicated equipment in most cases. Complex sample preparation means that continuous measurements are often not possible without complicated devices such as microfluidic chips. The presently disclosed device has been purposively devised to be simple and cheap to implement.

### SUMMARY

The inventors have found that the working inter-relationship between the features of the device allows for a finely tuned system suitable for monitoring oxidative stress markers. In this regard, the present relates to a device that may continuously monitor in-vivo concentrations of oxidative stress markers such as protein carbonyls. Further, the in-vivo device may measure dynamic changes in oxidative stress markers. Additionally, this small calibrated device may provide critical information about oxidative stress levels, and accordingly may be used to improve outcomes for people living with chronic disease and age-related disorders, as well as provide crucial redox balance information for athletes and livestock, embryologists and fertility experts and ultimately improve understanding about the mechanisms affecting overall health. Additionally, this device may be able to perform time resolved measurements that show the dynamic changes of protein carbonylation in a living sample, including cells, humans, animals, plants. Dynamic patterns in these measurements may be dependent on metabolic and developmental stages of the samples. Advantageously, the device does not require that the sample be further prepared, thus avoiding complex sample preparation making the device easier to use in the field (eg, high through put lab environment or doctor's surgery).

The present invention provides a device for monitoring oxidative stress in a sample, comprising:
a) a substrate;
b) a layer coated on the substrate; and
c) a compound having a moiety which is responsive to an oxidative stress marker in the sample, the compound doped within or on the surface of the layer;
   wherein the substrate and the layer are optically clear in the wavelength of about 400 nm to about 1000 nm.

The present invention also provides a device for monitoring oxidative stress in a sample, comprising:
a) an optically clear substrate;
b) an optically clear polymer layer, the polymer layer coated on the substrate; and
c) a fluorescent compound having a moiety which is responsive to an oxidative stress marker in the sample, the fluorescent compound doped within the polymer layer;
   wherein the substrate and the polymer layer are optically clear in the wavelength of about 400 nm to about 1000 nm.

In some embodiments, the moiety on the compound is a fluorescent compound responsive to a carbonyl moiety.

In some embodiments, the moiety on the compound is a fluorescent compound forms a reversible bond with a carbonyl moiety.

In certain some embodiments, the moiety on the compound is thiosemicarbazidyl, 1,3-dioxolanyl, or dithianyl.

In certain some embodiments, the compound is fluorescein-5-thiosemicarbazide (FTSC).

In some embodiments, the compound is doped within the layer up to about 10 wt % of the layer, and in ceratin embodiments a polymer. For instance, in certain embodiments the compound is doped within the layer (eg polymer layer) at about 0.1 wt % to about 10 wt %. In certain embodiments the compound is doped within the layer at about 0.5 wt % to about 10 wt %. In certain embodiments the compound is doped within the layer at about 0.1 wt % to about 5 wt %. In certain embodiments the compound is doped within the layer at about 0.1 wt % to about 4 wt %. In certain embodiments the compound is doped within the layer at about 0.1 wt % to about 3 wt %. In certain embodiments the compound is doped within the layer at about 0.1 wt % to about 2 wt %. In certain embodiments the compound is doped within the layer at about 0.1 wt % to about 1 wt %. In certain embodiments the compound is doped within the layer at about 0.1 wt %, at about 0.2 wt %, at about 0.3 wt %, at about 0.4 wt %, at about 0.5 wt %, about 0.6 wt %, about 0.7 wt %, about 0.8 wt %, about 0.9 wt %, or about 1 wt %.

In some embodiments, the layer comprises a straight chain polymer.

In some embodiments, the layer comprises a polymer selected from acrylate polymer, sulphonated polyetheretherketone, silk, polyacrylamide, vinylimidazole polymer, acrylonitrile butadiene styrene, photopolymer, or copolymers of the above.

In some embodiments, the layer comprises or is polymethyl methacrylate.

In some embodiments, the layer comprises or is an acrylate-based photopolymer, such as e-shell 300 acrylate-based photopolymer.

In certain some embodiments, the compound is fluorescein-5-thiosemicarbazide (FTSC) which is doped into or on the layer which is an acrylate-based photopolymer, such as e-shell 300 acrylate-based photopolymer.

In some embodiments, the layer has a thickness of up to 500 μm. For instance, in certain embodiments the layer has a thickness of about 5μm to about 500 μm, such as about 5 μm to about 400 μm, about 5 μm to about 300 μm, about 5 μm to about 200 μm, about 5 μm to about 100 μm, about 5 μm to about 50 μm, about 5 μm to about 40 μm, about 5 μm to about 30 μm, about 5 μm to about 20 μm, or about 5 μm to about 10 μm.

In some embodiments, the layer is coated at an end of the substrate.

In some embodiments, the layer coats the entire surface of the substrate.

In some embodiments, an emitted electronic or optical signal from the compound is detectable at an uncoated end of the substrate, for instance fluorescence or phosphorescence.

In some embodiments, the substrate is selected from a glass fibre, polymer fiber, microfluidic chip, glass or polymer slide, glass or polymer pipette.

The present also provides a device for monitoring oxidative stress in a sample, comprising:
a) a substrate;
b) an acrylate-based polymer layer, the layer coated on the substrate; and
c) a compound having a thiosemicarbazide moiety which is responsive to a carbonyl moiety in the sample, the compound doped within or on the surface of the polymer layer;
   wherein the substrate and the layer may be optically clear in the wavelength of about 400 nm to about 1000 nm.

The present invention also provides a device for monitoring oxidative stress in a sample, comprising:
a) an optically clear substrate;
b) an optically clear acrylate polymer layer, the polymer layer coated on the substrate; and
c) a fluorescent compound having a thiosemicarbazide moiety which is responsive to a carbonyl moiety in the sample, the fluorescent compound doped within the polymer layer;
   wherein the substrate and the polymer layer are optically clear in the wavelength of about 400 nm to about 1000 nm.

The present also provides a method of making a device for monitoring oxidative stress in a sample, including:
a) mixing a monomer with a compound to form a mixture, the monomer for forming an optically clear polymer and the compound having a moiety which is responsive to an oxidative stress marker in the sample;
b) contacting the mixture with a substrate; and
c) polymerising the mixture on the substrate for forming a layer coated on the substrate;
   wherein the substrate and the layer may be optically clear in the wavelength of about 400 nm to about 1000 nm.

The present invention also provides a method of making a device for monitoring oxidative stress in a sample, including:
a) mixing a monomer with a fluorescent compound to form a mixture, the monomer for forming an optically clear polymer and the fluorescent compound having a moiety which is responsive to an oxidative stress marker in the sample;
b) contacting the mixture with an optically clear substrate; and
c) polymerising the mixture on the substrate for forming a polymer layer coated on the substrate;
   wherein the substrate and the polymer layer are optically clear in the wavelength of about 400 nm to about 1000 nm.

In some embodiments, the mixing step comprises vortexing, sonicating or a combination thereof.

In some embodiments, the monomer is selected from a acrylate-based liquid photo-reactive photopolymer or methyl methacrylate (MMA).

In some embodiments, the polymerisation step comprises irradiating the mixture with a light at a wavelength of about 300 nm to about 600 nm. In some embodiments the light is at a wavelength of about 310 nm to 580 nm, 320 nm to 570 nm, 330 nm to 560 nm, 340 nm to 550 nm, 350 nm to 540 nm, 360 nm to 530 nm, 370 nm to 520 nm, 380 nm to 510 nm, or 390 nm to 500 nm.

In some embodiments, the polymer layer is coated at an end of the substrate.

In some embodiments, the method further includes connecting an uncoated end of the substrate to a light source and detector for measuring the electronic or optical signal, such as fluorescence or phosphorescence.

The present also provides a method of monitoring oxidative stress in a sample, including:
a) contacting a device as disclosed herein with the sample;
b) detecting an electronic or optical signal from the device, the signal being generated in response to an oxidative stress marker in the sample; and
c) quantifying the signal compared to a control signal.

The present invention also provides a method of monitoring oxidative stress in a sample, including:
a) contacting a device as disclosed herein with the sample;
b) detecting a fluorescence signal from the device, the fluorescence signal being generated in response to an oxidative stress marker in the sample; and
c) quantifying the fluorescence signal compared to a control signal.

In some embodiments, the response time of the device is less then about 60 sec, for monitoring dynamic changes in oxidative stress in the sample.

In some embodiments, the method is for use in in-vivo monitoring of oxidative stress and dynamic patterns.

### BRIEF DESCRIPTION OF THE DRAWINGS

Embodiments of the present invention will now be described, by way of non-limiting example, with reference to the drawings in which:

FIG. 1 is a schematic representation of a device according to an embodiment of the invention;

FIG. 2 is a schematic representation of a device according to another embodiment of the invention;

FIG. 3 is a plot of fluorescence intensity measured from a device when placed in a solution of β-hydroxybutyrate (BHB);

FIG. 4 is a plot showing an exemplary response from the device with decreasing concentrations of BHB every ten minutes;

FIG. 5 is a plot showing an exemplary response from 3D printed needle fitted with the device in three locations of pig skin;

FIG. 6 is a plot of fluorescence intensity against time of BHB at different concentrations;

FIG. 7 is a plot showing an exemplary response from the device in various locations of a live mouse;

FIG. 8 is a plot of fluorescence intensity against time, of in vivo use of the device in 3 different mice;

FIG. 9 is a plot of fluorescence intensity against time of in vivo use of the device in an exemplary mouse model;

FIG. 10 is a plot showing an exemplary response from the device in various locations of a wheat plant;

FIG. 11 illustrates (a) the setup of measurement system on wheat plant and (b) the device positioned inside a wheat seed;

FIG. 12 is a plot showing the measurement of protein carbonylation levels inside live wheat seed every minute for 8 days;

FIG. 13 is a plot showing the data from FIG. 12 fitted onto a kinetic model;

FIG. 14 illustrates (a) the setup of a young wheat plant before measurement starts, (b) the device inside a wheat stem at lower node with some noticeable yellowing of the leaves starting, and (c) the plant with device after 17 days;

FIG. 15 is a plot showing measurements of protein carbonylation levels inside live wheat stem node every minute for 17 days; and

FIG. 16 is a plot showing the detailed measurements from days 11-13 in FIG. 15.

### DETAILED DESCRIPTION

The present invention is predicated on the discovery that the working inter-relationship between the features of the device allows for a finely tuned system suitable for monitoring oxidative stress markers. In this regard, and as will be described herein, the features of the device act synergistically together such that the device may continuously monitor in-vivo concentrations of oxidative stress markers and may measure dynamic changes in oxidative stress markers.

The device of the present is for monitoring oxidative stress in a sample. The device comprises a substrate, a layer coated on the substrate (eg polymer layer), and a compound (eg fluorescent or phosphorescent) having a moiety which is responsive to an oxidative stress marker in the sample. The compound may be doped within the layer or be put on the surface of the layer.

In some embodiments, the substrate is optically clear. In other embodiments, the layer is optically clear. In other embodiments, the substrate and the layer are optically clear in the wavelength of about 400 nm to about 1000 nm. In this regard, the optic clarity of a substance may be quantified by, among other parameters, the substance's transparency and reflective index. Transparency is how easily a light may penetrate a substance and may be measured by, for example, a ASTM test (Standard Test Method for Haze and Luminous Transmittance of Transparent Plastics) which measures the amount of light that may make its way through a substance. The higher the value, the more light may get through and the clearer a substance is. This transmittance may for example be in the range of UV, visible and infrared wavelength (100 nm to 100 μm). Preferably, transmittance is in the visible wavelength of about 380 nm to about 740 nm. Refractive index is a measure of the ratio of the velocity of light in a vacuum to its velocity in a specified substance. Attenuation of light refers to the reduction in the light intensity as it travels through a substance, usually due to absorption or scattering of photons. The less light is attenuated through a substance, the clearer it is.

In other embodiments, the substrate and the layer are optically clear in the wavelength of about 400 nm to about 1000 nm. In other embodiments, the substrate and the layer are optically clear in the wavelength of about 400 nm to about 900 nm. In other embodiments, the substrate and the layer are optically clear in the wavelength of about 400 nm to about 800 nm. In other embodiments, the substrate and the layer are optically clear in the wavelength of about 400 nm to about 700 nm.

The optically clear substrate and layer advantageously allows the emitted electrical or optical signal to be transmitted to a receiver for quantification and analysis. In addition, the emitted electrical or optical signal is not significantly attenuated or altered and hence may result in an accurate measurement (i.e. close to actual measurement value). In this regard, at least 95% of the emitted signal is detected by a detector coupled to the substrate. In other embodiments, at least 90%, at least 85%, at least 80%, at least 75%, at least 70%, at least 60%, at least 50%, at least 40%, at least 30% or at least 20% of the emitted signal is detected.

In some embodiments, when the substrate and/or the layer is optically clear, the substrate and/or the layer has a light transmittance in the wavelength of about 300 nm to about 800 nm of more than about 90%. In other embodiments, the light transmittance is more than about 91%, about 92%, about 93%, about 94% or about 95% although the skilled person would understand that the layer, in certain embodiments, may not have transmittance this high.

Haze is measured as the percentage of incident light scattered by more than 2.5° through the substrate and/or the layer. Some factors responsible for light scattering is surface roughness, inhomogeneity and impurity. In some embodiments, the haze is less than about 15%, about 10%, about 9%, about 8%, about 7%, or about 5%, although the skilled person would understand that the layer, in certain embodiments, may not be characterized with haze this low.

The device functions by producing a signal in the presence of an oxidative stress marker. In some embodiments, the signal is an emitted fluorescence. This fluorescence may be produced by a fluorescent compound. The fluorescent compound is a fluorescent chemical compound that may re-emit light upon light excitation, and typically contain several combined aromatic groups, or planar or cyclic molecules with several π bonds. The compound may be inherently fluorescent, or may be fluorescent when the oxidative stress marker couples or binds to the compound.

For the compound to respond to the oxidative stress marker, the compound has at least a moiety which is responsive to an oxidative stress marker in the sample. In some embodiments, the oxidative stress marker is a protein carbonyl or a carbonyl moiety. The carbonyl may be an aldehyde moiety or a ketone moiety. In this regard, in some embodiments, the moiety on the compound is responsive to a carbonyl moiety.

The moiety on the compound may respond to the oxidative stress marker by forming a bond with the oxidative stress marker. The bond may be a covalent bond or a physical bond. For example, physical bonding such as electrostatic interaction, hydrogen bonding, Van der Waals interaction may be used to allow the compound to respond to the presence of the oxidative stress marker. The inventors have found that, in any case, the bonding is preferentially reversible. This allows the device to have a dynamic function and respond to the fluctuations in concentration of the oxidative stress marker over time.

In some embodiments, the moiety on the compound forms a reversible covalent bond with a carbonyl moiety. This allows the compound, and hence the device, to be regenerated and re-used for another measurement. Advantageously, by controlling the reversibility of the bond, the dynamic changes of oxidative stress marker in a sample may be measured accurately.

In some embodiments, when the oxidative stress marker is a carbonyl moiety, the moiety on the compound may be selected from thiosemicarbazidyl, 1,3-dioxolanyl, or dithianyl

The inventors have found that the compound may be further tuned to improve the performance of the device. For example, solubility of compound may influence its retention in the layer. If the compound is hydrophilic or polar, the compound may not be retained well in the layer and the device may lose its functionality over time. The electron cloud density of the compound may also be influenced by its interaction with the polymer layer.

In some embodiments, the compound is fluorescein-5-thiosemicarbazide (FTSC).

The compound is doped within or put on the surface of the layer. In this regard, the compound is doped within the layer up to about 10 wt % of the layer, or bonded to the surface of the layer. In some embodiments, the compound is present in the layer at less than 10 wt % of the layer. At higher concentrations, the inventors have found that the compound is likely to leach out from the layer. Further, higher concentrations of compound may result in self-quenching, which translates to a non-linear calibration plot with a plateau. This limits the accuracy of measurement of oxidative stress marker at high concentrations. In certain embodiments the compound is doped within the layer at about 0.1 wt %, at about 0.2 wt %, at about 0.3 wt %, at about 0.4 wt %, at about 0.5 wt %, about 0.6 wt %, about 0.7 wt %, about 0.8 wt %, about 0.9 wt %, about 1 wt %, about 2 wt %, about 3 wt %, about 4 wt %, about 5 wt %, about 6 wt %, about 7 wt %, about 8 wt % or about 9 wt %. In certain embodiments the compound is doped within the layer at about 0.1 wt % to about 10 wt %. In certain embodiments the compound is doped within the layer at about 0.5 wt % to about 10 wt %. In certain embodiments the compound is doped within the layer at about 0.1 wt % to about 5 wt %. In certain embodiments the compound is doped within the layer at about 0.1 wt % to about 4 wt %. In certain embodiments the compound is doped within the layer at about 0.1 wt % to about 3 wt %. In certain embodiments the compound is doped within the layer at about 0.1 wt % to about 2 wt %. In certain embodiments the compound is doped within the layer at about 0.1 wt % to about 1 wt %.

As mentioned, the moiety on the compound responds to the oxidative stress marker. In this regard, the moiety on the compound should preferentially not interact with the layer. The moiety responsive to the oxidative stress marker on the compound may preferentially not form a bond (covalent or physical) with the layer. This allows the moiety on the fluorescent compound to be available to bond to the oxidative stress marker such that the compound may respond.

The polymer layer comprises a matrix of polymer. In this regard, the density and crosslinking of the polymer may influence inclusion of the compound. For example, a highly crosslinked polymer may create a tight network such that the oxidative stress marker or compound of a particular size may be ‘squeezed out’ from the matrix. A polymer with a low crosslink or density may not be able to retain the compound within the polymer layer for prolong and repeated use.

In some embodiments, the polymer layer comprises a straight chain polymer. The inventors have found that straight chain polymers allow for an acceptable matrix density which traps the compound, does not hinder its property. It also allows for an acceptable amount of compound to be doped within the layer. The polymer may have a molecular weight of about 1.2 g/cm³.

As mentioned, the electron cloud density of the compound may also be influenced by its interaction with the layer. In this regard, the functionality of the layer may affect the signal. For example, nonpolar compound may not work in a hydrophilic environment of the layer. When a hydrophobic layer is used, the device is found not to perform as efficiently. It is found that in this case, the oxidative stress marker, which is polar, may still interact (albeit to a lesser extend) with the compound within or on the layer.

In some embodiments, the layer comprises a polymer selected from acrylate polymer, (such as Polymethyl methacrylate), or sulphonated polyetheretherketone, silk, polyacrylamide, vinylimidazole polymer, acrylonitrile butadiene styrene, photopolymer, or copolymers of the above.

In some embodiments, the layer comprises Polymethyl methacrylate.

The thickness of the layer may be controlled to provide a calibrated measureable response. With a thicker layer, more compound may be incorporated into the device. In some embodiments, the layer has a thickness of up to 500 μm. In other embodiments, the thickness is about 5 μm to about 500 μm, such as about 5 μm to about 400 μm, about 5 μm to about 300 μm, about 5 μm to about 200 μm, about 5 μm to about 100 μm, about 5 nm to about 90 μm, about 5 nm to about 80 μm, about 5 nm to about 70 μm, about 5 nm to about 60 μm, about 5 nm to about 50 μm, about 5 nm to about 40 μm, about 5 nm to about 30 μm, about 5 nm to about 20 μm, about 5 nm to about 10 μm, or about 5 nm to about 1 μm.

In some embodiments, the layer is coated at an end of the substrate. In this regard, the other end of the substrate is not coated with the layer. In some embodiments, an emitted signal from the compound is detectable at an uncoated end of the substrate. In this way, the emitted signal may travel from the coated end of the substrate to the uncoated end of the substrate. This allows the emitted signal to be accurately transported to a detector, which may be remote from the layer coated end of the substrate. This advantageously allows the design of the device to be more versatile and allows for a compact design.

In some embodiments, the layer coats the entire surface of the substrate. In such embodiments, the substrate may be a microfluidic device.

Surface roughness and reflections of the substrate may create transmission losses. Surface reflections may be caused by specular reflection, which is the normal reflection from a smooth surface, and diffuse reflection, which is dependent on the surface flatness of the substrate. The transmission loss as a result of surface roughness or embedded particles may also be due to how the substrate is formed. This transmission loss (light scattering) may be attributed to ‘haze’.

In some embodiments, the substrate is selected from a glass fibre, polymer fiber, microfluidic chip, glass or polymer slide, glass or polymer pipette.

In some embodiments, the substrate has a transmission loss of less than about 90%, about 91%, about 92%, about 93%, about 94%, about 95%, or about 96%.

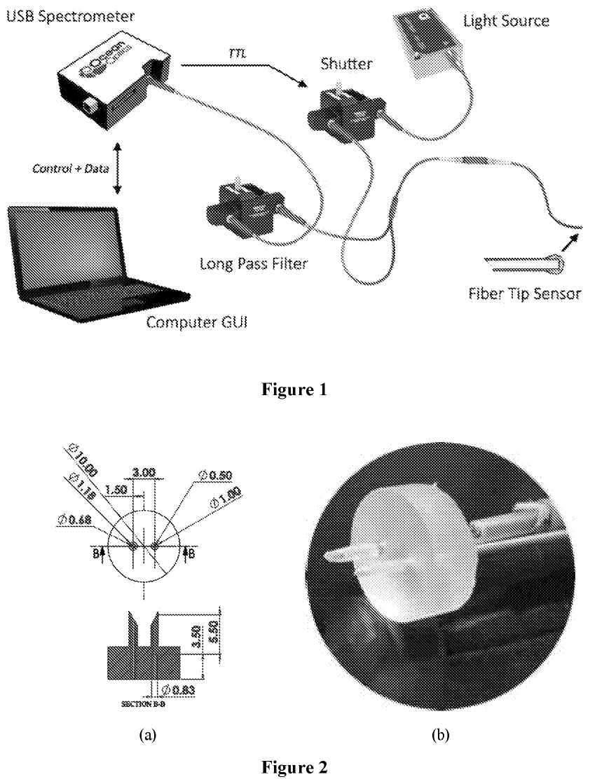

FIG. 1 illustrates a schematic representation of an embodiment of the present invention. The device for monitoring oxidative stress is a fiber tip sensor, which is connected to a light source via a shutter. The excitation light may travel to the fiber tip sensor due to the total internal reflection effect of the glass fiber. The shutter serves to select a particular time range which is suitable for exciting the fluorescent compound. The emitted fluorescence then travels from one coated end of glass fiber tip to the uncoated end to be detected by the detector which is housed in the spectrometer. The data may be processed by a computer and tabulated based on a calibration plot.

FIG. 2 illustrates another embodiment of the present, in which a 3D printed needle is fitted with the device for monitoring oxidative stress (an optical fiber tip sensor). This optical fiber based in-vivo biosensor allows for piercing of a surface of a sample and is capable of providing information about dynamic changes in oxidative stress levels, redox balance, and diurnal patterns.

In another embodiment, the device may be positioned on a microfluidic chip. In this regard, the layer is coated on a chip surface (substrate). The emitted signal may be detected by a detector which is, for example, positioned at an angle to the excitation source. For example, the detector may be positioned at about 90° from the excitation source. The detector may be positioned either at a face adjacent to the layer or at a face adjacent to the substrate. As mentioned, when the layer and/or the substrate are optically clear, the emitted signal may be received by the detector without it being attenuated.

Accordingly, the present invention also provides a device for monitoring oxidative stress in a sample, comprising:
a) a substrate;
b) an acrylate-based polymer layer, the polymer layer coated on the substrate; and
c) a compound having a thiosemicarbazide moiety which is responsive to a carbonyl moiety in the sample, the fluorescent compound doped within or on the surface of the polymer layer;
   wherein the substrate and the layer may be optically clear in the wavelength of about 400 nm to about 1000 nm.

Accordingly, the present invention also provides a device for monitoring oxidative stress in a sample, comprising:
a) an optically clear substrate;
b) an optically clear acrylate polymer layer, the polymer layer coated on the substrate; and
c) a fluorescent compound having a thiosemicarbazide moiety which is responsive to a carbonyl moiety in the sample, the fluorescent compound doped within the polymer layer;
   wherein the substrate and the polymer layer are optically clear in the wavelength of about 400 nm to about 1000 nm.

The present invention also provides a method of making a device for monitoring oxidative stress in a sample, including:
a) mixing a monomer with a compound to form a mixture, the monomer for forming an optically clear polymer and the compound having a moiety which is responsive to an oxidative stress marker in the sample;
b) contacting the mixture with an substrate; and
c) polymerising the mixture on the substrate for forming a layer coated on the substrate;
   wherein the substrate and the layer may be optically clear in the wavelength of about 400 nm to about 1000 nm.

The present invention also provides a method of making a device for monitoring oxidative stress in a sample, including:
a) mixing a monomer with a fluorescent compound to form a mixture, the monomer for forming an optically clear polymer and the fluorescent compound having a moiety which is responsive to an oxidative stress marker in the sample;
b) contacting the mixture with an optically clear substrate; and
c) polymerising the mixture on the substrate for forming a polymer layer coated on the substrate;
   wherein the substrate and the polymer layer are optically clear in the wavelength of about 400 nm to about 1000 nm.

In some embodiments, the mixing step comprises vortexing, sonicating or a combination thereof. Vortexing involves a mass of fluid (such as a liquid) with a whirling or circular motion that tends to form a cavity or vacuum in the center of the circle and to draw toward this cavity or vacuum bodies subject to its action especially. Sonication uses sound waves to agitate the components in a solution. It converts an electrical signal into a physical vibration to mix solutions, accelerate the dissolution of a solid into a liquid and remove dissolved gas from liquids. The mixing step allows for the homogenisation of the compound within the polymer layer. In particular, the inventors have found that by starting from a monomer instead of a pre-formed polymer, better dispersion of the compound within the polymer layer may be obtained.

In some embodiments, the monomer for forming the polymer is selected from acrylate-based liquid photo-reactive photopolymer, or MMA.

Examples of commercial sources of monomers include e-shell 300 acrylate-based photopolymer (EnvisionTEC), and methyl methacrylate (Sigma-Aldrich).

When the mixture is contacted with the substrate, the inventors have further found that how the mixture is contacted with the substrate may affect the optical clarity of the layer. In this regard, surface roughness of the layer may create transmission losses. Such surface roughness may be a result of an uneven coating or a non-homogenous distribution of the compound in the polymer layer. Poor contact of the layer with the substrate may also result in reduced optical clarity.

In some embodiments, the mixture is contacted with the substrate by dip coating. In other embodiments, the mixture is contacted with the substrate by spin coating.

The monomer may be polymerised by way of free radical polymerisation. In this regard, the initiation of the polymerisation process may be by thermal decomposition, photolysis, redox reactions, persulfates dissociation, use of an ionising radiation, plasma, sonication or by electrochemical means. In some embodiments, the polymerisation step comprises irradiating the mixture with a light at a wavelength of about 300 nm to about 600 nm. In some embodiments the light is at a wavelength of about 310 nm to 580 nm, 320 nm to 570 nm, 330 nm to 560 nm, 340 nm to 550 nm, 350 nm to 540 nm, 360 nm to 530 nm, 370 nm to 520 nm, 380 nm to 510 nm, or 390 nm to 500 nm. In other embodiments, the mixture is irradiated with a UV light at a wavelength of about 250 nm to about 400 nm. In other embodiments, the mixture is irradiated for about 1 min to about 30 min. In other embodiments, the polymerisation step comprises a heat source.

In some embodiments, the layer is coated at an end of the substrate. In some embodiments, the method further includes connecting an uncoated end of the substrate to a light source and/or detector for measuring the signal. The light source is for excitation of the compound and the detector is for measuring the emitted signal. The light source may be further tuned by passing it through a shutter, for selecting a range of excitation times. The detector may be a spectrometer or photodetector. A long pass filter may be connected between the device and the detector for filtering out excitation light from measured signal.

The present inventions also provides a method of monitoring oxidative stress in a sample, including:
a) contacting a device as disclosed herein with the sample;
b) detecting an electronic or optical signal from the device, the signal being generated in response to an oxidative stress marker in the sample; and
c) quantifying the signal compared to a control signal.

The present invention also provides a method of monitoring oxidative stress in a sample, including:
a) contacting a device as disclosed herein with the sample;
b) detecting a fluorescence signal from the device, the fluorescence signal being generated in response to an oxidative stress marker in the sample; and
c) quantifying the fluorescence signal compared to a control signal.

The signal is compared to a control signal for quantifying the amount of oxidative stress marker in the sample. For example, the control signal may be a calibrated plot of signal intensity with respect to the concentration of carbonyl moiety in a solution. Alternatively, the control signal may be a measurement or an average of measurements from sample(s) determined to be normalised. Alternatively, the control signal may be a signal measurement or an average of measurements from sample(s) determined to be normalised. Alternatively, the control signal may be a signal measurement from another compound which is added to the device or measurement system to act as a reference, and example of which may be a nanoparticle or a fluorophore.

In some embodiments, the response time of the device is up to about 60 sec, for monitoring dynamic changes in oxidative stress in the sample. The response time is calculated from the time the excitation source excites the compound, to the time the emitted signal is detected. In other embodiments, the response time is up to about 50 sec, about 40 sec, about 30 sec, about 25 sec, about 20 sec, about 15 sec, about 10 sec, about 5 sec, about 1 sec, about ½ sec, about ⅕ sec, or about 1/10 sec.

In some embodiments, the method is for use in in-vivo monitoring of oxidative stress and dynamic patterns. The device may be used for assisting people living with chronic disease such as diabetes, cardiovascular disease, infertility and fetal health. The device may be used for assisting athlete training by providing an individual's stress levels and recovery. The device may also be used for assisting agricultural analysis by providing plant stress levels and dynamic patterns.

Accordingly, the present invention also provides a method of in-vivo monitoring oxidative stress, including:
a) contacting a device as disclosed herein with an organism;
b) detecting an electronic or optical signal from the device, the signal being generated in response to an oxidative stress marker in the sample; and
c) quantifying the signal compared to a control signal.

Accordingly, the present invention also provides a method of in-vivo monitoring oxidative stress, including:
a) contacting a device as disclosed herein with an organism;
b) detecting a fluorescence signal from the device, the fluorescence signal being generated in response to an oxidative stress marker in the sample; and
c) quantifying the fluorescence signal compared to a control signal.

In certain embodiments, the organism is an organism in need thereof of in-vivo monitoring of oxidative stress.

### EXAMPLES

### Device Fabrication (Fiber Tip Sensor)

4 mg of fluorescein-5-thiosemicarbazide (Cayman Chemical; FTSC) was added to 0.5 ml e-shell 300 acrylate-based photopolymer (EnvisionTEC). This photopolymer is Class-IIa biocompatible, and FTSC is known to reversibly bind to protein carbonyls which induces fluorescence (495/517 nm Ex./Em. Max). The mixture was vortexed for 1 minute then sonicated for 10 minutes then vortexed for a further 1 minute. A 600 μm patch fiber (Thorlabs; M29L05) was cut in half and stripped back to expose the solid glass core which was then cleaved. The tip of the glass core was functionalized by dipping the tip into the FTSC doped photopolymer for 1 second. The FTSC doped photopolymer left behind at the fiber tip was then exposed to 488 nm laser light (Integrated Optics MatchBox CW) at 142 μW by coupling the 488 nm laser light into fiber via the subminiature version A (SMA) connector for 30 seconds. Next, the tip of the fiber was dip cleaned by quickly dipping into a solution of 50/50 ratio acetone and isopropanol (IPA) followed by quickly dipping into a solution of only IPA. The last step of the procedure was to expose the fiber tip to 80 mW of 405 nm laser light (Integrated Optics MatchBox CW) by coupling the 405 nm laser light into the SMA connector for 5 minutes. This procedure left behind a layer of FTSC doped polymer at tens of micrometers thick on the tip of the cleaved M29L05 patch fiber to create a fiber tip oxidative stress sensor (tip sensor).

### Measurement Setup

As illustrated in FIG. 1, the fully fiber coupled setup is comprised of a 488 nm laser light source (Integrated Optics MatchBox CW) and shutter to send a 0.1 sec pulse of 130 μW excitation light to the tip sensor via a bifurcated fiber bundle (Thorlabs; RP21). The back reflected signal was coupled back into the bifurcated fiber bundle, passed through a 488 nm long-pass edge filter (Semrock; BLP01-488R-25), and characterized using an Ocean Optics QE Pro spectrometer.

### β-hydroxybutyrate (BHB) Characterization Solutions

0.5 mM BHB stock solution was prepared by re-constituting the contents of a β-hydroxybutyrate Standard vial (Cayman Chemical Company; 700192), which contains a lyophilized powder of DL-hydroxybutyrate, with 2 ml of β-Hydroxybutyrate Assay Buffer (Cayman Chemi-cal Company; 700191). The reconstituted solution is stable for six hours on ice. The standard curve was obtained immediately by using the setup to measure the stock solution and then reducing concentrations of the BHB solution. These reducing concentrations where prepared by adding an additional appropriate volume of β-Hydroxybutyrate Assay Buffer to the 0.5 mM BHB stock solution to obtain a stepwise change in concentration, as shown in Table 1.

TABLE 1
BHB solution preparation.

| | Buffer | Total |
|---|---|---|
| Concentration | Added | Volume |
| 0.5 mM | — | 200 μl |
| 0.4 mM | 50 μl | 250 μl |
| 0.3 mM | 83 μl | 333 μl |
| 0.2 mM | 167 μl | 500 μl |
| 0.1 mM | 500 μl | 1000 μl |

### 3D Printed Needles

Hollow microneedles were produced from Class-IIa biocompatible e-shell 300 acrylate-based photopolymer (EnvisionTEC) using an Envisiontec Perfactory stereolithography machine. The needle design is shown in FIG. 2(a) and the final 3D printed microneedle is shown in FIG. 2(b).

### The Quadratic Equation for Protein Carbonyl Kinetics

The Michaelis-Menten model is the one of the simplest and best-known approaches to enzyme kinetics. It takes the form of an equation relating reaction velocity to substrate concentration for a system where a substrate C (reactive oxygen species) binds to enzyme P to form an enzyme-substrate complex PC (protein carbonyl), which then reacts irreversibly to generate a product E and to regenerate the free enzyme P. This system may be represented schematically as follows:

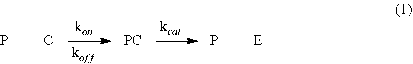

where kₒₙ is the bimolecular association rate constant of enzyme-substrate binding; k_{off} is the unimolecular rate constant of the PC complex dissociating to regenerate free enzyme and substrate; and kcat is the unimolecular rate constant of the PC complex dissociating to regenerate a free enzyme P and product E. The units for kₒₙ is concentration⁻¹time⁻¹, and k_{off} and k_{cat} have units of time⁻¹. Also, by definition the dissociation binding constant of the PC complex, K_{d} is given by:

$${K}_{d} = \frac{{k}_{off}}{{k}_{on}} \qquad ( 2 )$$

and so has units of molar concentration (M). In the case of reactive oxygen species binding to proteins P we assume that P+C→PC is an irreversible reaction and therefore k_{off}=0, so we have:

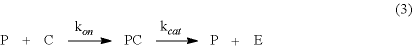

and we define the rate constant:

$${K}_{m} = \frac{{+ {k}_{cat}}^{0}}{{k}_{on}} = \frac{{k}_{cat}}{{k}_{on}} \qquad ( 4 )$$

which also has units of molar concentration (M). The rate of change differential equations may be written as:

$$\frac{d [ C ]}{dt} = - {k}_{on} [ P ] [ C ] \qquad ( 5 )$$

$$\frac{d [ P ]}{dt} = {k}_{cat} [ P C ] - {k}_{on} [ P ] [ E ]$$

$$\frac{d [ E ]}{dt} = {k}_{cat} [ P C ]$$

$$\frac{d [ P C ]}{dt} = {k}_{on} [ P ] [ C ] - {k}_{cat} [ P C ]$$

where [P], [C], [E], and [PC] represent molar concentrations of the enzyme, reactive oxygen species, product and protein carbonyl complex, respectively.

Assuming that [PC] protein carbonyl levels achieve steady state, then:

$$\begin{matrix}\frac{d [ PC ]}{dt} = 0 & ⟹ & {k}_{on} [ P ] [ C ] = {k}_{cat} [ PC ]\end{matrix} \qquad ( 6 )$$

Given that at steady state the free enzyme concentration [P] equals total enzyme concentration [Pₜ] minus [PC] and that free reactive oxygen species concentration [C] equals total reactive oxygen species concentration [Cₜ] minus [PC], we have:

$${k}_{on} ( [ {P}_{t} ] - [ P C ] ) ( [ {C}_{t} ] - [ P C ] ) = {k}_{cat} [ P C ] \qquad ( 7 )$$

$$∴ ( [ {P}_{t} ] - [ P C ] ) ( [ {C}_{t} ] - [ P C ] ) = \frac{{k}_{cat}}{{k}_{on}} [ P C ] = {K}_{m} [ P C ]$$

$$∴ {[ P C ]}^{2} - ( [ {P}_{t} ] + [ {C}_{t} ] ) [ P C ] + [ {P}_{t} ] [ {C}_{t} ] = {K}_{m} [ P C ]$$

$$∴ {[ P C ]}^{2} - ( [ {P}_{t} ] + [ {C}_{t} ] + {K}_{m} ) [ P C ] + [ {P}_{t} ] [ {C}_{t} ] = 0$$

which has the quadratic form:

ax²+bx+c=0 (8)

where

a=1

b=−([Pₜ]+[Cₜ]+Kₘ)

c=[Pₜ][Cₜ] (9)

### Device Characterisation

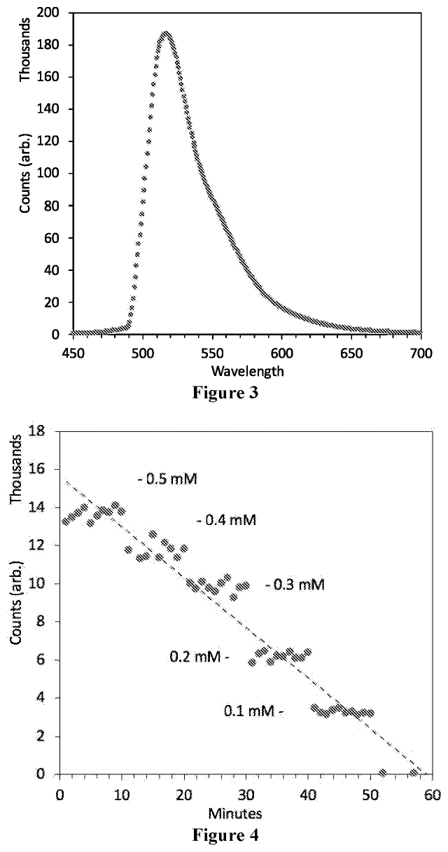

As shown in FIG. 3, the device is responsive to carbonyl moiety (BHB ketone). The emitted fluorescence at 517 nm is due to fluorescein-5-thiosemicarbazide. The intensity of the fluorescence peak at 517 nm was found to be proportional to the concentration of protein carbonyls within a sample. FIG. 4 shows the unadjusted peak counts measured from one pulse every minute with the sensor in solution of β-Hydroxybutyrate standard with decreasing concentrations every 10 minutes (the last measurement was per-formed in buffer only).

### In-Vivo Porcine Skin Measurements

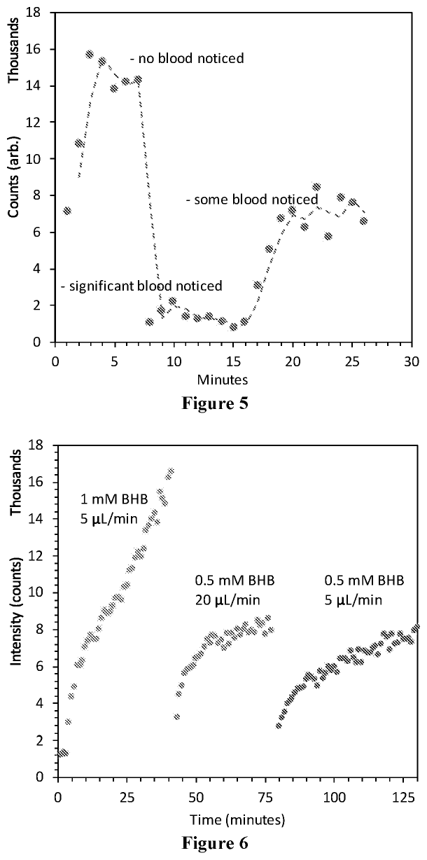

In-vivo measurements of protein carbonylation using the biosensor were performed on pig-skin. The biosensor device was inserted into fresh pig-skin (3 hours old) sourced from an abattoir and measurements were recorded every minute. FIG. 5 shows the results of the response from the biosensor while being subdermal in three locations of the pig-skin. The signal was an order of magnitude higher in regions of the skin where blood was not noticeably present. The same experiment was repeated on nine day old pig-skin, where the biosensor showed very low intensity; orders of magnitude less signal compared to the 3 hours old sample.

### In-Vivo Live Mouse Measurements

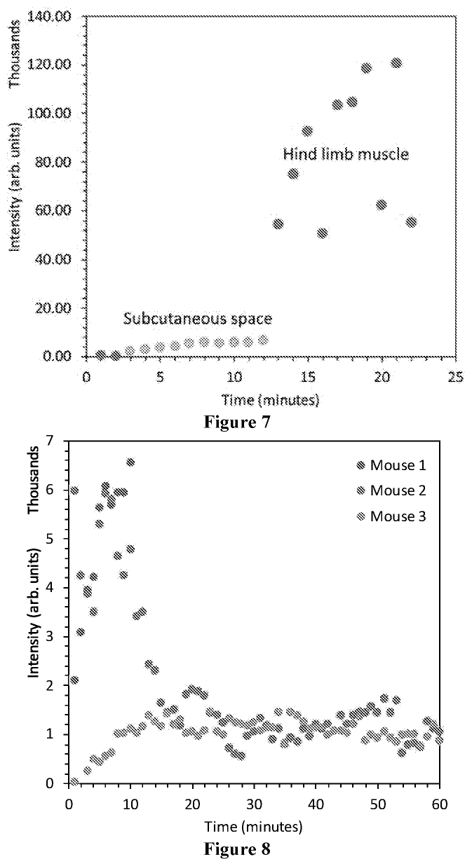

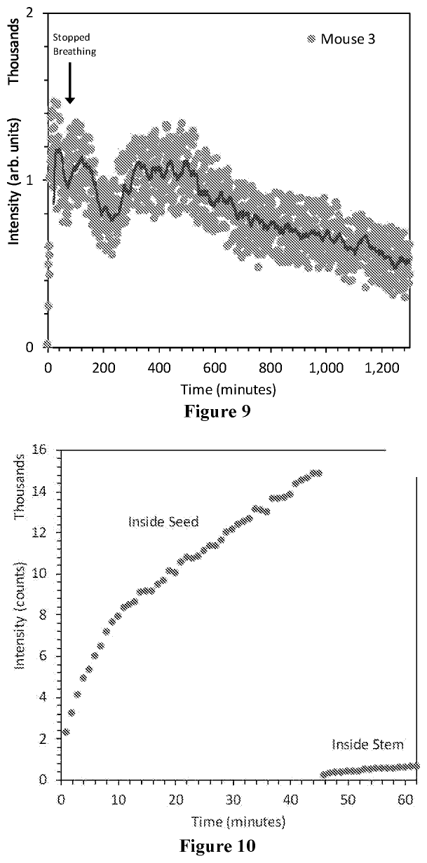

The biosensor was used to measure protein carbonylation levels in an anesthetized live mouse. In this case, the polymer microneedle was replaced by a stainless-steel needle. FIG. 7 shows a detectable and relatively steady concentration of the analyte in the subcutaneous space under the skin. In the hind limb muscle, where there was blood, the levels were much higher; they appeared to increase over the ten minutes of measurement.

### In-Vivo Wheat Plant Measurements

The biosensor, without any needle, was placed within the seed and stem areas of a wheat plant. FIG. 10 shows that the signal continued to increase while the sensor was in the plant, with an order of magnitude higher signal within the seed compared to the stem.

### In-Vivo measurements of Protein Carbonylation in Wheat Seed

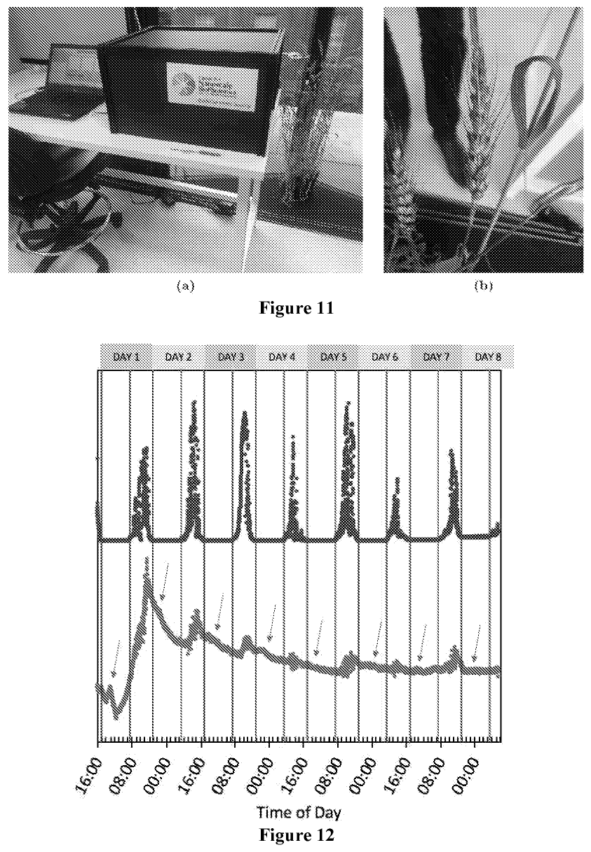

The fully fibre coupled setup was placed near a north facing window where the plant could have maximal exposure to sun light. FIG. 11a shows the setup and FIG. 11b shows the optical fibre with tip sensor inside one of the plants seeds. Measurements of Protein Carbonylation levels were taken inside live wheat seed every minute for 8 days while the plant was in its drying cycle. FIG. 12 shows the results from these measurements, where the green points show the intensity coming from the optical fibre sensor tip which correlates to protein carboxylation levels and the violet points shows the relative intensity of ambient light on the plant. The dark red and light red lines indicate sunset and sunrise times respectively. The blue arrows show a previously unobserved feature that occurs each day after sunset related to the plant's metabolic circadian rhythm. The circadian rhythm of the wheat plant at the seed follows a cycle that includes increased oxidative stress levels during the plants diurnal cycle between ˜11:00 am (˜4 hours after sunrise) and ˜3:15 pm (˜2.5 hours before sunset) and decreasing levels during the plants nocturnal cycle for the rest of the day.

### Fitting Seed Data to Protein Carbonyl Kinetics Model

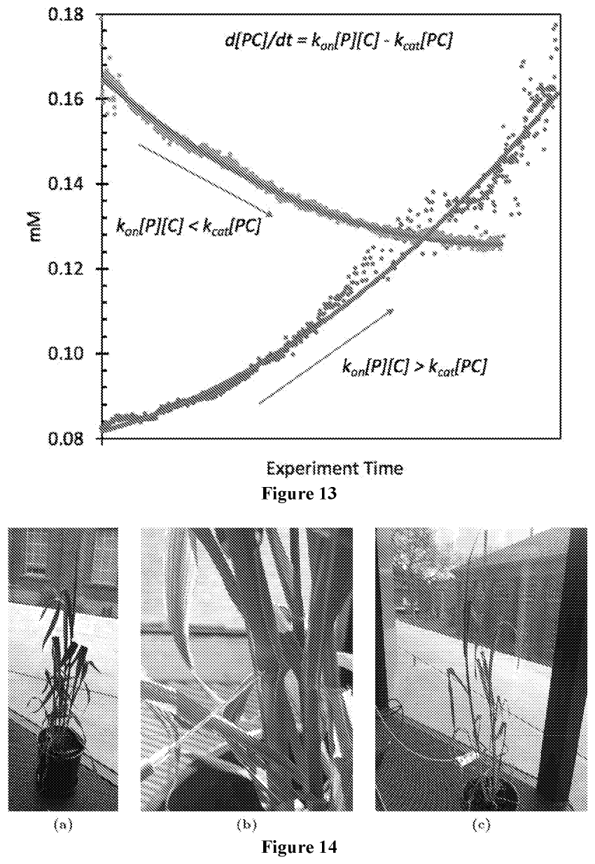

The seed data (FIG. 12) from the time the signal started to increase on day one (around 1 am) until the signal started to increase on day two (around 9 am) was plotted as two data sets; data during the increasing segment and data during the decreasing segment. This data, shown in FIG. 13, was normalised against a concentration vs intensity curve (produced using the calibration curve of the sensor in known concentrations of β-hydroxybutyrate) on the vertical axis and normalised to ensure the fitted quadratic model from Eq. (7) (shown in red) had an a value equal to 1.

The b and c values of the quadratic model (Eq. (7)) with the results fitted as shown in FIG. 13 lead to the set of equations,

Increasing Segment

−([Pₜ]+[Cₜ]+Kₘ)=3.95×10⁻³

[Pₜ][Cₜ]=8.18×10⁻⁵ (10)

Decreasing Segment

−([Pₜ]+[Cₜ]+Kₘ)=−1.25×10⁻²

[Pₜ][Cₜ]=1.65×10⁻⁴ (11)

Solving for these equations we get the concentration of total proteins ([Pₜ]) and ROS ([Cₜ]) involved in the reaction as
Increasing Segment: [Pₜ]=2.0 M and [Cₜ]=4.1×10⁻⁵ M
Decreasing Segment: [Pₜ]=2.0 M and [Cₜ]=8.2×10⁻⁵ M
   which indicates that the plant was twice as efficient at reducing ROS levels in the seed during nocturnal cycle compared to the plants diurnal cycle.

### In-Vivo Measurements of Protein Carbonylation in Wheat Stem

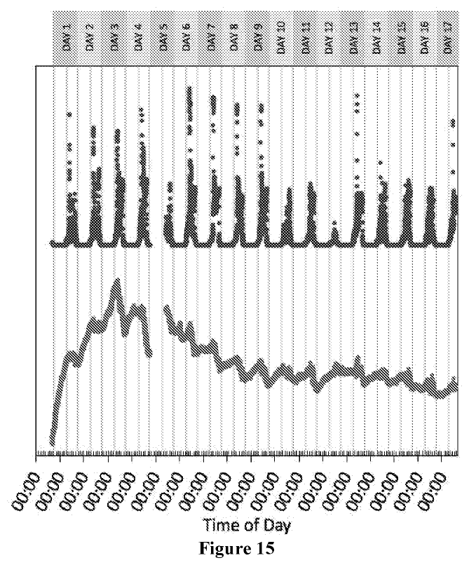

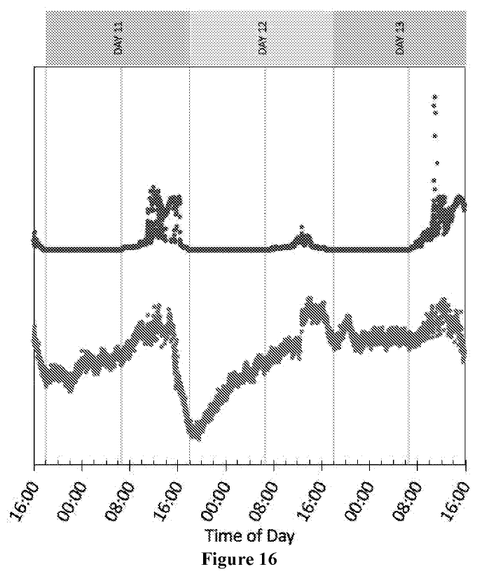

A new young wheat plant was dug up from the field and placed in the controlled environment lab for 5 days prior to starting measurements, shown in FIG. 14a. At first this new plant had green leaves without any noticeable browning or yellowing. After the 5 days in the lab, and before measurements started, this plant started to show some signs of leaf yellowing. The fibre tip sensor was placed into the lower node of the new younger wheat plant. FIG. 14b shows the optical fibre with tip sensor inside one of the nodes, and some noticeable leaf yellowing. Measurements of Protein Carbonylation levels were taken inside live wheat node every minute for 17 days, at which time the plant had not grown and had significantly increased leaf yellowing and browning as shown in FIG. 14c. FIG. 15 shows the results from these measurements, where the green points show the intensity coming from the optical fibre sensor tip which correlates to protein carboxylation levels and the violet points shows the relative intensity of ambient light on the plant. The dark red and light red lines indicate sunset and sunrise times respectively.

The circadian rhythm of this plant measured at the node follows an inverse pattern compared to the results in the seed from the previous plant. Starting every day at around sunset, the increases occur during the plants nocturnal cycle while the decreases occur during the plants diurnal cycle starting with oscillations from ˜11 am-1 pm and then steady decrease until sunset. On day 12 there was no noticeable decrease in protein carbonyl levels during the diurnal cycle, with much less light during that day compared to others because of thick cloud and heavy rain. Instead, the protein carbonyl levels noticeably increased within a few minutes at ˜1:00 pm and then came back down to pre-1:00 pm level between ˜4-6 pm when the sun set. Subsequently, on day 13 the protein carbonyl levels stayed reasonably steady overnight, during the nocturnal cycle, and increased in the morning before decreasing again from ˜1:00 pm with normal light levels. There are also some interesting small oscillation features that occur during the nocturnal cycles.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A device for monitoring oxidative stress in a sample, comprising:
a) a substrate;
b) a layer coated on the substrate; and
c) a compound having a moiety which is responsive to an oxidative stress marker in the sample, the compound doped within or on the surface of the layer;
wherein the substrate and the layer are optically clear in the wavelength of about 400 nm to about 1000 nm.

2. The device according to claim 1, wherein the moiety on the compound is responsive to a carbonyl moiety, and preferably fluorescent

3. The device according to claim 1 or 2, wherein the moiety on the compound forms a reversible bond with a carbonyl moiety.

4. The device according to any of claims 1 to 3, wherein the moiety on the compound is thiosemicarbazide, or dithianyl.

5. The device according to any of claims 1 to 4, wherein the compound is fluorescein-5-thiosemicarbazide (FTSC).

6. The device according to any of claims 1 to 5, wherein the compound is doped within layer up to about 10 wt % of the layer, preferably 0.1 wt % to 1 wt % of the layer.

7. The device according to any of claims 1 to 5, wherein the compound is doped on the surface of the layer up to about 10 wt % of the layer, preferably 0.1 wt % to 1 wt % of the layer.

8. The device according to claim 6 or 7 wherein the compound is FTSC and is doped at about 0.1 wt % to about 1 wt % of the layer.

9. The device according to any of claims 1 to 8, wherein the layer is a polymer layer, preferably a polymer which comprises a straight chain polymer.

10. The device according to claim 9, wherein the polymer layer comprises a polymer selected from acrylate polymer, sulphonated polyetheretherketone, silk, polyacrylamide, vinylimidazole polymer, acrylonitrile butadiene styrene, photopolymer, or copolymers of the above.

11. The device according to any of claim 9, wherein the polymer layer comprises acrylated-based photopolymer, and preferably e-shell 300 acrylate-based photpolymer.

12. The device according to any of claims 1 to 8, wherein the layer is a glass layer.

13. The device according to any of claims 1 to 12, wherein the layer has a thickness of up to 500 μm, preferably up to 100 μm.

14. The device according to any of claims 1 to 13, wherein the layer is coated at an end of the substrate.

15. The device according to any of claims 1 to 14, wherein the layer coats the entire surface of the substrate.

16. The device according to any of claims 1 to 15, wherein an emitted electrical or optical signal from the compound is detectable at an uncoated end of the substrate.

17. A device for monitoring oxidative stress in a sample, comprising:
a) an optically clear substrate;
b) an optically clear acrylate polymer layer, the polymer layer coated on the substrate; and
c) a fluorescent compound having a thiosemicarbazide moiety which is responsive to a carbonyl moiety in the sample, the fluorescent compound doped within the polymer layer;
wherein the substrate and the polymer layer are optically clear in the wavelength of about 400 nm to about 1000 nm.

18. A method of making a device for monitoring oxidative stress in a sample, including
a) mixing a monomer with a fluorescent compound to form a mixture, the monomer for forming an optically clear polymer and the fluorescent compound having a moiety which is responsive to an oxidative stress marker in the sample;
b) contacting the mixture with an optically clear substrate; and
c) polymerising the mixture on the substrate for forming a polymer layer coated on the substrate;
wherein the substrate and the polymer layer are optically clear in the wavelength of about 400 nm to about 1000 nm.

19. The method according to claim 18, wherein the mixing step comprises vortexing, sonicating or a combination thereof.

20. The method according to claim 18 or 19, wherein the monomer is selected from a acrylate-based liquid photo-reactive photomonomer.

21. The method according to anyone of claims 18 to 19 wherein the fluorescent compound is FTSC.

22. The method according to any of claims 18 to 21, wherein the polymerisation step comprises irradiating the mixture with a light at a wavelength of about 300 nm to about 600 nm.

23. The method according to any of claims 18 to 22, wherein the polymer layer is coated at an end of the substrate.

24. The method according to any of claims 18 to 23, further including connecting an uncoated end of the substrate to a light source and detector for measuring the fluorescence.

25. A method of monitoring oxidative stress in a sample, including:
a) contacting a device as defined in anyone of claims 1 to 17 with the sample;
b) detecting a fluorescence signal from the device, the fluorescence signal being generated in response to an oxidative stress marker in the sample; and
c) quantifying the fluorescence signal compared to a control signal.

26. The method according to claim 25, wherein the response time of the device is up to about 60 sec, for monitoring dynamic changes in oxidative stress in the sample.

27. The method according to claim 25 or 26, for use in in-vivo monitoring of oxidative stress and dynamic patterns.

28. The method according to anyone of claims 25 to 27 for use in intracytoplasmic sperm injection (ICSI) technology, plant cell or animal cell physiological studies, or determining human performance.
